(19) United States
(12) Patent Application Publication
(10) Pub. No.: US 2013/0261744 A1
(43) Pub. Date: Oct. 3, 2013

(54) IMPLANTABLE OPHTHALMIC DEVICE WITH AN ASPHERIC LENS

(71) Applicants: Amitava Gupta, Roanoke, VA (US); Nicholas Wooder, Roanoke, VA (US); Ronald D. Blum, Roanoke, VA (US); Rudy Mazzocchi, Roanoke, VA (US); Urban Schnell, Roanoke, VA (US)

(72) Inventors: Amitava Gupta, Roanoke, VA (US); Nicholas Wooder, Roanoke, VA (US); Ronald D. Blum, Roanoke, VA (US); Rudy Mazzocchi, Roanoke, VA (US); Urban Schnell, Roanoke, VA (US)

(73) Assignee: Elenza, Inc., Roanoke, VA (US)

(21) Appl. No.: 13/701,432

(22) PCT Filed: May 31, 2011
(86) PCT No.: PCT/US2011/038597

§ 371 Date: Jun. 10, 2013

Related U.S. Application Data
(60) Provisional application No. 61/350,344, filed on Jun. 1, 2010.
(60) Provisional application No. 61/361,653, filed on Jul. 6, 2010.
(60) Provisional application No. 61/384,336, filed on Sep. 20, 2010.

(51) Int. Cl. A61F 2/16

(52) U.S. Cl. CPC A61F 2/1616; A61F 2/1624
USPC 623/6.22; 623/6.23

(57) ABSTRACT

Implantable ophthalmic devices with aspheric lenses and dynamic electro-active elements offer excellent depth of field and image quality while providing high optical throughput. An exemplary implantable ophthalmic device includes an aspheric lens with a negative spherical aberration that varies with radius. The aspheric lens can have peak optical powers at its geometric centers surrounded by a region of varying optical power (with varying slope) that extends radially from its center. When implanted, these aspheric lenses provide an incremental optical power that varies as a function of pupil diameter, which changes with object distance, for viewing far, intermediate, and near objects. The aspheric lens may also bonded or integrally formed with a spherical lens that provides fixed optical power for viewing far objects and/or a dynamic electro-active element that with two or more states (e.g., on and off) for increasing the effective optical power when viewing near objects.

### CROSS-REFERENCE TO RELATED PATENT APPLICATIONS

This application claims the benefit of U.S. Provisional Application No. 61/350,344, filed Jun. 1, 2010, and entitled “Modeling Retinal Image Quality of an Aspheric IOL with an Embedded Dynamic Aperture”; U.S. Provisional Application No. 61/361,653, filed Jul. 6, 2010, and entitled “Intermediate Vision Provided by an Aspheric IOL with an Embedded Dynamic Aperture”; and U.S. Provisional Application No. 61/384,336, filed on Sep. 20, 2010, and entitled “An Intraocular Implant that comprises a Dynamic Focusing Element and a Static Aspheric Focusing Element.” Each of the above-referenced applications is incorporated herein by reference in its entirety.

### BACKGROUND

There are two major conditions that affect an individual's ability to focus on near and intermediate distance objects: presbyopia and pseudophakia. Presbyopia is the loss of accommodation of the crystalline lens of the human eye that often accompanies aging. In a presbyopic individual, this loss of accommodation first results in an inability to focus on near distance objects and later results in an inability to focus on intermediate distance objects. It is estimated that there are approximately 90 million to 100 million presbyopes in the United States. Worldwide, it is estimated that there are approximately 1.6 billion presbyopes. Pseudophakia is the replacement of the crystalline lens of the eye with an artificial lens, known as an intraocular lens (IOL), usually following surgical removal of the crystalline lens during cataract surgery. In a pseudophakic individual, the absence of the crystalline lens causes a complete loss of accommodation that results in an inability to focus on either near or intermediate distance objects. For all practical purposes, an individual will get cataracts if he or she lives long enough. Furthermore, most individuals with cataracts will have a cataract operation at some point in their lives. It is estimated that approximately 1.2 million cataract surgeries are performed annually in the United States.

The standard tools for correcting presbyopia are reading glasses, multifocal ophthalmic lenses, and contact lenses fit to provide monovision. Reading glasses have a single optical power for correcting near distance focusing problems. A multifocal lens is a lens that has more than one focal length (i.e., optical power) for correcting focusing problems across a range of distances. Multifocal optics are used in eyeglasses, contact lenses, and IOLs. Multifocal ophthalmic lenses work by means of a division of the lens's area into regions of different optical powers. Multifocal lenses may be comprised of continuous surfaces that create continuous optical power as in a Progressive Addition Lens (PAL). Alternatively, multifocal lenses may be comprised of discontinuous surfaces that create discontinuous optical power as in bifocals or trifocals. Contact lenses fit to provide monovision are two contact lenses having different optical powers. One contact lens is for correcting mostly far distance focusing problems and the other contact lens is for correcting mostly near distance focusing problems.

Aphakia can also be corrected using IOLs. Conventional IOLs are monofocal spherical lenses that provide focused retinal images for far objects (e.g., objects over two meters away). Generally, the focal length (or optical power) of a spherical IOL is chosen based on viewing a far object that subtends a small angle (e.g., about seven degrees) at the fovea. Typical patients require spherical IOLs with optical powers between about +10 diopters (D) and about +36 D. The most commonly required optical power is about +25 D or about +26 D. Unfortunately, all spherical surfaces, including spherical IOLs and the cornea, suffer from spherical aberration, which limits image quality.

Fortunately, aspheric IOLs can be used to treat presbyopia and aphakia without introducing spherical aberration. In fact, aspheric IOLs with constant negative spherical aberration can even be used to compensate spherical aberration introduced in the cornea. Although these conventional aspheric IOLs provide better images for a wider range of far object scenarios than spherical IOLs, conventional aspheric IOLs do not improve image quality for near or intermediate objects. It has always been always accepted that patients with aspheric IOLs would wear glasses to see near and intermediate objects.

Conventional approaches for correcting presbyopia and/or aphakia suffer other drawbacks as well, some of which are more severe than others. For example, while spectacle eyewear is capable of correcting one's vision for far, near and intermediate distances, this approach requires wearing a device that takes away from one's natural appearance. Also, in some cases, certain multifocal lenses may cause the user to perceive distortion and experience vertigo. Approaches for correcting presbyopia and/or aphakia that include the use of contact lenses can cause discomfort and can also result in one or more of: halos, doubling of vision, light scattering, glare, loss of contrast sensitivity, limited range of focus, and/or reduction of light hitting the retina. Approaches that include the use of IOLs can result in one or more of: light scattering, glare, halos, ghosting, loss of contrast sensitivity, limited range of focus, and/or reduction of light hitting the retina. These drawbacks, or compromises to one's vision, can be very problematic especially, by way of example only, when driving at night, driving in the rain, or working on a computer.

### SUMMARY

Embodiments of the technology disclosed herein include an implantable ophthalmic device with an aspheric optical element that has a negative spherical aberration, which is the variation in focal point with incoming ray height, that varies as a function of radius. The negative spherical aberration may be at a maximum at or near the optical center of the aspheric optical element. In some ophthalmic optical elements, the negative spherical aberration varies within a range of about 0.10 μm to about 5.0 μm of root-mean-square wavefront error across an exit pupil of 5.0 mm or less in diameter. The negative spherical aberration may also be substantially non zero (i.e., have an absolute value that is greater than zero) over a radius about 0.50 mm to about 2.5 mm centered about the geometric center of the aspheric optical element.

Further embodiments of the disclosed technology, the aspheric optical element has a sag that is continuous. As understood by those of skill in the art, the sag is the distance between the vertex of a reference sphere on the optical axis and the surface of the aspheric element at a given distance (radius) from the optical axis. In embodiments with continuous sags, the first and second derivatives of the sag with respect to the transverse dimensions (radius) may also be continuous.

When implanted in a patient's eye, exemplary aspheric optical elements may provides an average incremental optical power of about +0.25 D or less, or possibly about +0.10 D or less.

Implanted aspheric optical elements can provide a maximum incremental optical power of about +0.5 D to about +0.8 D. Illustrative implantable ophthalmic devices may also include a spherical optical element in optical communication with the aspheric optical element. The spherical optical element may have a base optical power of about +10 D to about +36 D, e.g., about +25 D or about +26 D.

Further embodiments of the implantable ophthalmic devices disclosed herein may include an electro-active element in optical communication with the aspheric optical element. Such electro-active elements may be switched or tuned between a first state with a first effective optical power and a second state with a second effective optical power. In some cases, the electro-active element has a first refractive index in the first state and a second refractive index in the second state; in other cases, the electro-active element has a first transmissivity in the first state and a second transmissivity in the second state; in still other cases, both the refractive index and transmissivity may vary between or among states. Alternatively, or in addition, the electro-active element can act as an aperture with a first diameter in the first state and a second diameter in the second state. Regardless of its mechanism of action, the electro-active element can provide a first or effective optical powers is about +0.5 D to about +2.5 D.

An exemplary implantable ophthalmic device with an electro-active element may also include a processor operably coupled to the electro-active optical element and configured to switch or tune the electro-active optical element, e.g., between the first and second states. Such an implantable ophthalmic device may also include a sensor that is operably coupled to the processor and configured to provide an indication of pupil size to the processor, which may be configured to actuate the electro-active optical element in response to the indication of the pupil size. Such an illustrative implantable ophthalmic device may also include an antenna operably coupled to the processor and configured to transmit and receive data, and, optionally, at least one battery operably coupled to the processor and configured to provide power to the processor. The battery may be recharged via the antenna.

In alternative embodiments, an implantable ophthalmic device may include a spherical optical element, an aspheric optical element in optical communication with the spherical optical element, and an electro-active element in optical communication with the spherical and aspheric optical elements. The spherical optical element has a fixed optical power (e.g., about +10 D to about +36 D), whereas the aspheric optical element has an optical power that varies as a function of radius, and may be about +0.25 D or less. The electro-active element has at least two states, each with a different effective optical power, at least one which may be about +0.5 D to about +2.5 D.

Yet another embodiment includes an implantable ophthalmic device with an electro-active element, a spherical optical element, and an aspheric optical element with a sag whose first and second derivatives with respect to radius are continuous. The electro-active element, spherical optical element, and aspheric optical element may be in optical communication with each other, and the electro-active element can have a first state with a first effective optical power and a second state with a second effective optical power.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

The accompanying drawings, which are incorporated in and constitute a part of this specification, illustrate embodiments of the invention and together with the description serve to explain principles of the invention.

FIG. 1 shows a cross section of a healthy human eye.

FIG. 2 shows an exemplary implantable ophthalmic device that includes an aspheric optical element in optical communication with a spherical optical element and an electro-active element.

FIG. 3 illustrates the desired optical power, sag profile, sag, and shape of an exemplary aspheric optical element.

FIG. 4 is a plot of local incremental optic power versus radius for three different exemplary aspheric optical elements.

FIG. 5 is a plot of average incremental optical power versus pupil diameter for an exemplary aspheric optical element.

FIG. 6 shows an exemplary electro-active element.

FIGS. 7A and 7B include plots of modulation transfer functions for aspheric and standard intra-ocular lenses used with apertures of different sizes.

FIGS. 8A and 8B show images of objects at different distances for aspheric and standard intra-ocular lenses used with apertures of different sizes.

### DETAILED DESCRIPTION

Presently preferred embodiments of the invention are illustrated in the drawings. An effort has been made to use the same or like reference numbers to refer to the same or like parts.

### Overview of the Eye

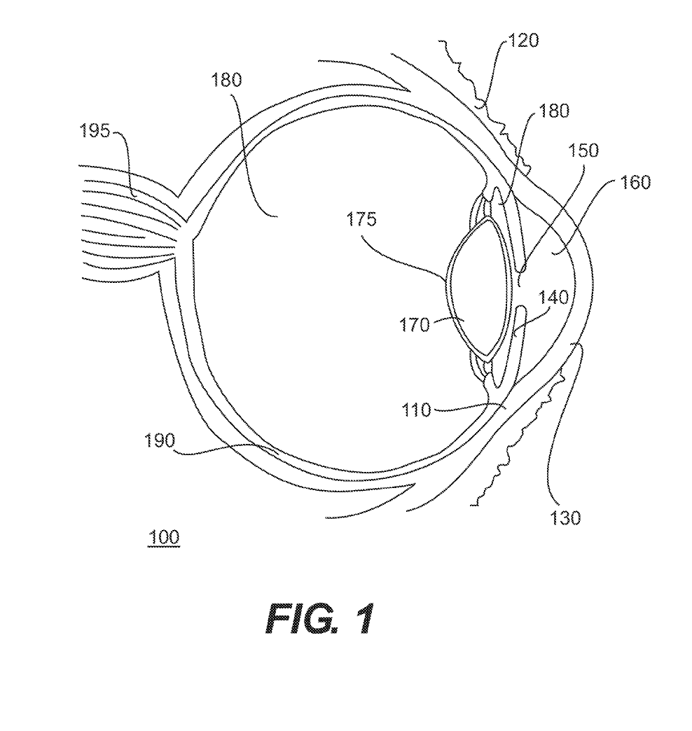

FIG. 1 shows a cross section of a healthy human eye 100. The white portion of the eye is known as the sclera 110 and is covered with a clear membrane known as the conjunctiva 120. The central, transparent portion of the eye that provides most of the eye's optical power is the cornea 130. The iris 140, which is the pigmented portion of the eye and forms the pupil 150. The sphincter muscles constrict the pupil and the dilator muscles dilate the pupil. The pupil is the natural aperture of the eye. The anterior chamber 160 is the fluid-filled space between the iris and the innermost surface of the cornea. The crystalline lens 170 is held in the lens capsule 175 and provides the remainder of the eye's optical power. The retina 190, which is separated from the back surface of the iris 140 by the posterior chamber 180, acts as the “image plane” of the eye and is connected to the optic nerve 195, which conveys visual information to the brain.

A healthy crystalline lens 170 is capable of changing its optical power such that the eye is capable of imaging objects at near, intermediate, and far distances to the front surface of the retina 190 in a process known as accommodation. Presbyopic individuals suffer from a loss of accommodation, which makes it difficult for them to focus on near objects; as their disease progresses, they eventually lose the ability to focus on intermediate objects as well. An aphakic individual has no crystalline lens and, therefore, cannot focus on object at near or intermediate distances.

### Overview of Ophthalmic Devices Implantable in Eye

The implantable ophthalmic devices disclosed herein include may be used to compensate for the degradation or loss of accommodation suffered by presbyopic individuals without dramatically reducing the amount of light transmitted to the retina. They may also be used to provide an accommodative response for aphakic individuals. Exemplary implantable ophthalmic devices include an aspheric optical element that, unlike other aspheric IOLs, has a negative spherical aberration that varies as a function of radius. When the device is implanted in the patient, the combination of variation in negative spherical aberration and the change in the patient's pupil size with object distance gives the patient's eye an effective optical power that changes with object distance. As a result, a patient with an exemplary implantable ophthalmic device can focus more easily on far, intermediate, and near objects instead of just on far objects.

Exemplary implantable ophthalmic devices may also include a spherical optical element and an electro-active element that acts as switchable lens or variable-diameter aperture. The spherical optical element has a constant optical power of about +10 D to about +36 D (e.g., +25 D or +26 D) for improving the patient's ability to see far objects (e.g., objects at distances greater than about two meters). The electro-active element act as a switchable or tunable lens or aperture for dynamically changing the depth of field (and the net optical power) of the device as a function of object distance to further enhance image quality of near and intermediate objects.

Exemplary implantable ophthalmic devices may be inserted or implanted in the anterior chamber or posterior chamber of the eye, into the capsular sac, or the stroma of the cornea (similar to a corneal inlay), or into the epithelial layer of the cornea (similar to a corneal onlay), or within any anatomical structure of the eye. Generally, the implanted ophthalmic devices may provide a visual acuity of no worse than 20/30 under photopic conditions at all object distances while maintaining best corrected distance visual acuity of 20/20 or better. Ideally, the eye of a patient implanted with an illustrative device should be no more than 1.0 D out of focus at all object distances, since a defocus of 1.0 D leads to an acuity of 20/30 or better when the natural depth of focus of the eye is taken into account.

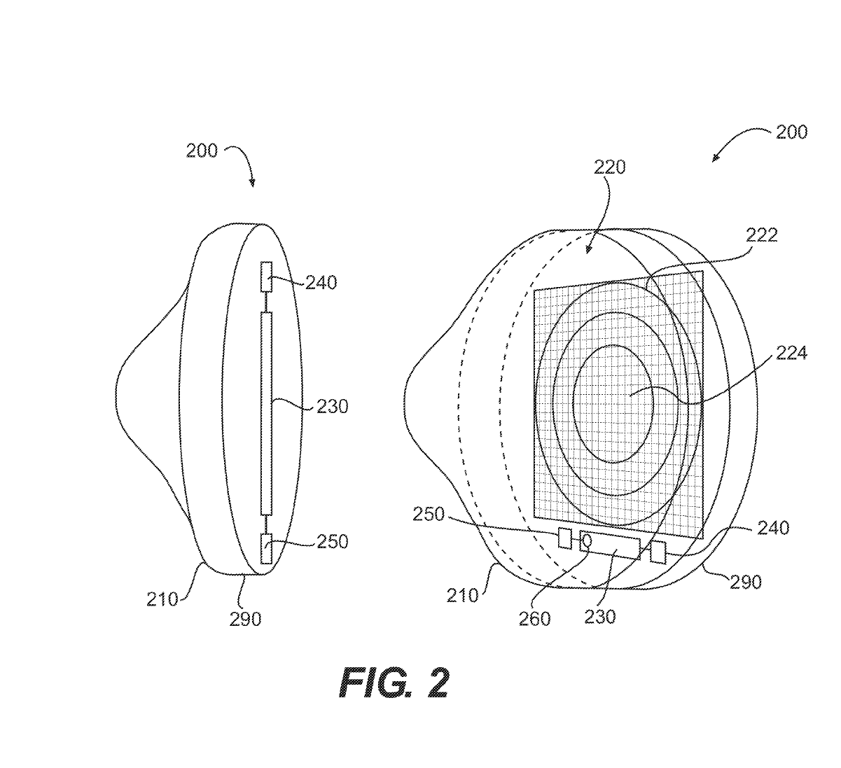

FIG. 2 shows an exemplary implantable ophthalmic device 200, which may be used as an intra-ocular lens (IOL), that can be used to compensate for degradation, loss, or absence of accommodation. (Although the device 200 shown in FIG. 2 has a circular area, other implantable ophthalmic devices may be square, rectangular, elliptical, etc.) The implantable ophthalmic device 200 includes an aspheric optical element 210, or aspheric lens, with a negative spherical aberration that varies as a function of radius. A spherical optical element 290, or spherical lens, with fixed optical power provides base compensation for viewing far objects. An electro-active element 220 embedded in or affixed to the aspheric optical element 210 or the spherical optical element 290 acts as a switchable lens and/or variable-diameter aperture that can be used to change the depth of field.

A processor 230, such as an application-specific integrated circuit (ASIC), can be used to control the electro-active element 220 based on estimates of an object distance. The estimate can be determined, for example, in response to signals from a sensor 240 that measures at least one physiological indication of the eye's natural accommodative response, including, but not limited, to changes in pupil size and/or ion concentration. More specifically, the processor 230 may estimate the object distance based on measurements from the sensor 260 of changes in the size of the natural pupil due to convergence of the eyes on a near or intermediate object.

One or more rechargeable batteries 250 coupled to the processor 220 provide power for the processor 220 and other electronic components in the implantable ophthalmic device 200. The device 200 also includes an antenna 260, which may be configured to receive either radio-frequency (rf) or optical signals for controlling or updating the processor 220, and may also be used to charge the batteries 250 as described below.

### Examples of Aspheric Optical Elements for Use in Implantable Ophthalmic Device

An aspheric optical element, also referred to as an aspheric lens or an asphere, is a rotationally symmetric optic whose radius of curvature varies radially from its center. Unlike spherical lenses, which have a constant radius of curvature, aspheric lenses have a radius of curvature that changes with distance from the optical axis. Aspheric lenses have shapes that have been traditionally defined by:

$$Z ( s ) = \frac{{Cs}^{2}}{1 + \sqrt{1 - ( 1 + k ) {C}^{2} {s}^{2}}} + {A}_{4} {s}^{4} + {A}_{6} {s}^{6} + {A}_{8} {s}^{8} + … , \qquad ( 1 )$$

where Z is the sag of the surface parallel to the optical axis, s is the radial distance from the optical axis, C is the curvature (i.e., the inverse of the radius), k is the conic constant, and Aₙ are weights for higher-order aspheric terms. When the aspheric coefficients are equal to zero, the resulting aspheric surface is considered to be a conic: for k=0, the conic surface is spherical (i.e., the lens is spherical rather than aspherical); for k>−1, the conic surface is ellipsoidal; for k=−1, the conic surface is paraboloidal; and for k=+1, it is hyperboloidal. The sag can also be described more precisely as

$$Z ( s ) = \frac{{C}_{bfs} {s}^{2}}{1 + \sqrt{1 - ( 1 + k ) {C}_{bfs}^{2} {s}^{2}}} + {u}^{4} {\sum }_{m = 0}^{M} {a}_{m} {Q}_{m}^{con} ( {u}^{2} ) , \qquad ( 2 )$$

where C_{bfs} is the curvature of the best-fit sphere, u=s/sₘₐₓ, Qₘ^{con} is the orthonormal basis of the asphere coefficients, and aₘ is a normalization term.

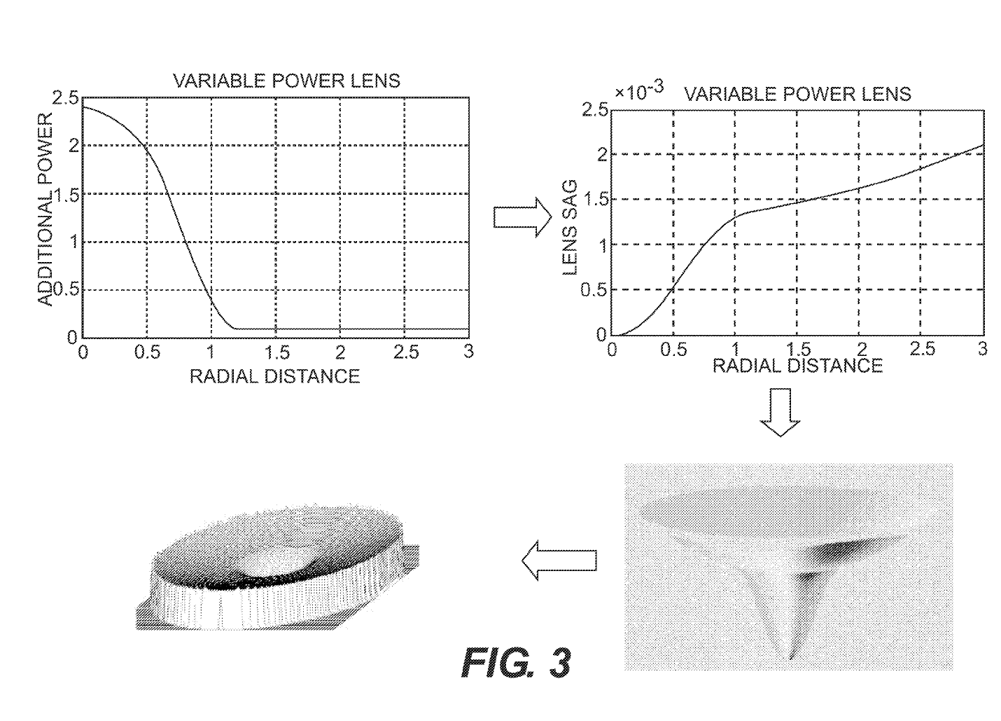

FIG. 3 illustrates the design process and shape of an illustrative aspheric optical element. First, the designer determines the desired optical power profile of the aspheric optical element as a function of radius as shown at upper left. As shown in FIG. 3, the optical power is at maximum at the center of the aspheric optical element. The optical power decreases smoothly with a slope that increases, then decreases over a central zone, which may extend over a radius of about 1.25 mm to about 2.5 mm (e.g., 1.5 mm as shown in FIG. 3), before reaching or asymptotically approaching a constant value. This change in slope of the optical power also manifests itself as a variable spherical aberration. Visual acuity at far object distances (e.g., object distances of four meters or more) may be compromised if the power of the static aspheric optical element exceeds 1.50 D and extends over a central radius of more than 3.0 mm.

Next, the designer translates the desired optical power profile into a sag, or surface profile, of the lens as a function of radius as shown at the upper right of FIG. 3. The sag reflects the desired optical power profile: it equals zero at the center, increases smoothly with increasing slope over a narrow central zone, reaches an inflection point at a radius about 1.0 mm, increases with decreasing slope over a radius of about 1.0 mm to about 1.75 mm. Beyond a radius of about 1.75 mm, the sag follows a circular arc or a parabolic arc. Rotating the sag profile about the y axis of the plot at the upper right of FIG. 3 yields the surface profile at the bottom right of FIG. 3, which can be used to create the aspheric optical element shown at the bottom left of FIG. 3.

As shown in the upper right of FIG. 3, exemplary aspheric optical elements have sags that are continuous with respect to the transverse dimension, r. (Aspheric lens are rotationally symmetric with respect to the optical axis, so there is no need to specify the azimuth.) In other words, the sag does not have any discontinuities, such as kinks or ledges, which produce undesired reflections and/or optical scattering. Exemplary sags also have continuous first and second derivatives with respect to the transverse dimension. Selecting the sag to have a continuous first derivative with respect to r suppresses or eliminates undesired double images due to prismatic effects. Selecting the sag to have a continuous second derivative with respect to r reduces or eliminates undesired discontinuities in magnification (e.g., discontinuities that cause straight lines to appear wavy).

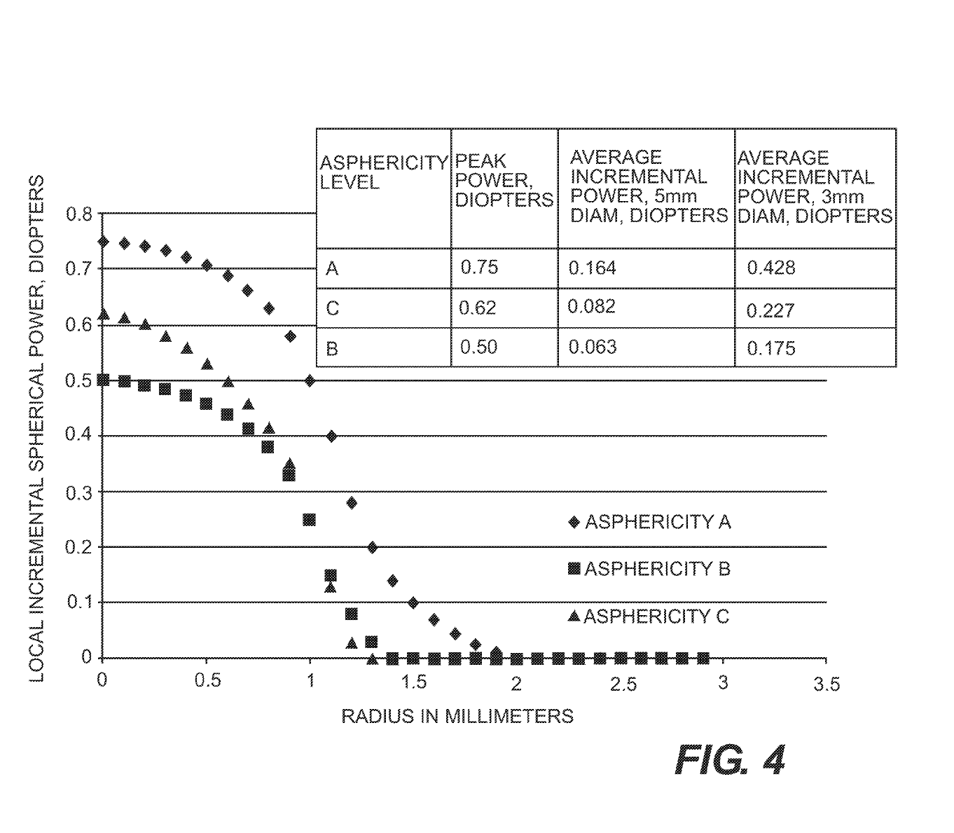

FIG. 4 is a plot of local incremental optical power versus radius for three different aspheric optical elements implanted in the eye. The local incremental power represents the optical power added by a particular position of an aspheric optical element implanted in a patient's eye. The exact amount of added incremental optical power depends, in part, on the shape of the aspheric optical element, its index of refraction, and its position within the eye. As shown in FIG. 4, the peak incremental optical power is at the center of aspheric optical element (i.e., at r=0) and is within a range of about 0.5 D to about 0.8 D, e.g., about 0.5 D, about 0.62 D, or about 0.75 D. The local incremental optical power decreases over a radius of about 1.25 mm to about 2.5 mm until it reaches zero at the outer region of the aspheric optical element.

The patient experiences the additional optical power of the aspheric optical element as an average incremental optical power, which is the local incremental optical power averaged across the portion of the aspheric optical element that projects an image onto the retina. In exemplary devices, the average incremental power is under about 0.25 D, e.g., about 0.10 D or less. When the patient focuses on far objects, the pupil opens, allowing the retina to receive an image projected through a larger portion of the aspheric optical element. As a result, the average incremental power decreases. When the patient focuses on intermediate or near objects, the pupil closes, so the retina receives an image projected through a smaller portion (i.e., just the more radically aspheric portion) of the aspheric optical element, resulting in a increase in the average incremental optical power.

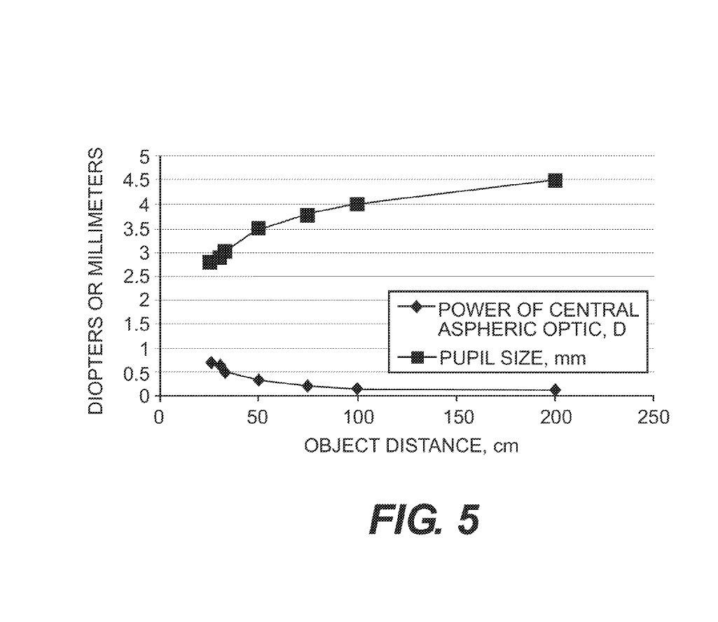

FIG. 5 and TABLE 1 show changes average incremental optical power as a function of pupil diameter. As explained above, the average incremental optical power decreases with increasing pupil size, which correlates with object distance, before asymptotically approaching an imperceptible amount of optical power—under about 0.125 D. Similarly, the average incremental optical power of an aspheric optical element with asphericity A described in FIG. 4 varies from about 0.16 D to about 0.43 D for a change in pupil diameter from about 5 mm to about 3 mm as shown in FIG. 4. Thus, an implantable ophthalmic device with an exemplary aspheric optical element provides variable optical power for focusing on near, intermediate, and far objects without moving parts or multiple optical elements with different focal lengths.

TABLE 1
Design of the Aspheric Optical Element

| Object Distance | Assumed Pupil | Added Optical |
|---|---|---|
| (cm) | Diameter (mm) | Power (D) |
| 200 | 4.5 | <0.125 |
| 100 | 4.0 | 0.15 |
| 75 | 3.8 | 0.20 |
| 50 | 3.5 | 0.35 |
| 33 | 3.0 | 0.50 |
| 30 | 2.9 | 0.65 |
| 25 | 2.5 | 0.75 |

Aspheric lenses can also be used with variable-diameter apertures, such as those described below, be used to provide both good image quality and high optical throughput. Increasing the f-number (F/#) of the eye by “stopping down” the eye with an variable-diameter aperture improves image quality as described in U.S. Pat. No. 7,926,940 to Blum et al., which is incorporated herein by reference in its entirety, but also reduces the amount of light incident on the retina. On the other hand, when using aspheric lenses, additional aberration correction makes it possible to design high throughput (low F/#) systems while simultaneously maintaining good image quality. The image degradation from a higher throughput design can be sustained because a slight tradeoff in image quality can still outperform a spherical lens used with an aperture. The additional aberration correction of aspheric lenses also eliminates the need for additional optical elements, such as those is multi-element lenses, for high-quality imaging of far, intermediate, and near objects.

Illustrative aspheric lenses can be made of optical glass, plastic, thermoplastic resins, thermoset resins, a composite of glass and resin, or a composite of different optical grade resins or plastics. For example, aspheric lenses can be made using injection-molded plastic or resin. Molten plastic is injected into an appropriately shaped aspheric mold and allowed to harden before being removed. The electro-active element, processor, sensor, batteries, and other elements may embedded in a plastic aspheric lens during injection molding or affixed to a plastic aspheric lens before the lens has fully hardened. If necessary, the electronic components may be coated with an appropriate heat-resistant material so that they won't be damaged during manufacturing. The position of the electro-active element with respect to the aspheric optical element can be adjusted during the molding process and may be chosen depending on each element's respective optical power. For example, the electro-active element can be positioned in the front, the center, or the rear of an aspheric optical element or spherical optical element. Alternatively, the aspheric lens can be made using conventional glass grinding and polishing techniques, and the electro-active element, spherical optical element, and other components can be bonded or sealed together with the aspheric lens.

Aspheric optical elements (and the implantable ophthalmic as a whole) can flexible and/or have folding designs for easer implantation in the eye. For example, the lens and device may fold about one or more fold lines for insertion, then unfold about the fold line(s) once properly positioned within the eye. Rigid components may be disposed on either side of the fold line(s) for ease of insertion. For more, see U.S. application Ser. No. 12/017,858, entitled “Flexible Electro-Active Lens,” and U.S. application Ser. No. 12/836,154, entitled “Folding Designs for Intraocular Lenses,” each of which is incorporated herein by reference in its entirety.

### Examples of Spherical Optical Elements for Use in Implantable Ophthalmic Device

Illustrative implantable ophthalmic devices may also include spherical lenses or spherical optical elements, which are components with spherically shaped surfaces that cause light to converge or diverge (i.e., a spherical lens is capable of focusing light). Exemplary spherical lenses have a fixed optical power of about +10 D to about +36 D (e.g., about +20 D to about +30 D, or about +25 or +26 D). Suitable spherical lenses may be refractive, diffractive, or a combination thereof. They may be concave, convex, or planar on one or both surfaces—graded index (GRIN) lenses are also suitable. They may be made of optical glass, plastic, thermoplastic resins, thermoset resins, a composite of glass and resin, or a composite of different optical grade resins or plastics. Exemplary spherical optical elements may be bonded or affixed to other components in the implantable ophthalmic device, including the aspheric optical element and/or the electro-active element. They may also be formed integral to or with the aspheric optical element and/or the electro-active element.

Exemplary spherical lenses may be either conventional or non-conventional. A conventional lens corrects for conventional errors of the eye including lower order aberrations such as myopia, hyperopia, presbyopia, and regular astigmatism. A non-conventional lens corrects for non-conventional errors of the eye including higher order aberrations that can be caused by ocular layer irregularities or abnormalities. The spherical lens may be a single-focus (monofocal) lens or a multifocal lens, such as a Progressive Addition Lens or a bifocal or trifocal lens.

### Examples of Electro-Active Elements for Use in Implantable Ophthalmic Device

As used herein, the term “electro-active element” refers to a device with an optical property that is alterable as a function of space and/or time by the application of electrical energy. The alterable optical property may be, for example, optical power, which, for a lens, is the reciprocal of the focal length; refractive index (retardance); optical transmittance (transmissivity); diffraction efficiency; aperture size, shape, or position; or any combination thereof. An electro-active element may be constructed from two substrates and an electro-active material disposed between the two substrates. The substrates may be shaped and sized to ensure that the electro-active material is contained within the substrates and cannot leak out. One or more electrodes may be disposed on each surface of the substrates that is in contact with the electro-active material. The electro-active element may include or be coupled to a power supply operably connected to a controller. The controller may be operably connected to the electrodes by way of electrical connections to apply one or more voltages to each of the electrodes. When electrical energy is applied to the electro-active material by way of the electrodes, the electro-active material's optical property may be altered. For example, when electrical energy is applied to the electro-active material by way of the electrodes, the electro-active material's index of refraction may be altered, thereby changing the optical power of the electro-active element.

The electro-active element may be embedded within or attached to a surface of an aspheric optical element and/or a spherical optical element to form an electro-active lens. Alternatively, the electro-active element may be embedded within or attached to a surface of an optic which provides substantially no optical power to form an electro-active optic. In such a case, the electro-active element may be in optical communication with an aspheric optical element and/or a spherical optical element, but separated or spaced apart from or not integral with the aspheric optical element and/or the spherical optical element. The electro-active element may be located in the entire viewing area of the aspheric optical element and/or the spherical optical element or in just a portion thereof, e.g, near the top, middle or bottom portion of the lens or optic. The electro-active element may be capable of focusing light on its own.

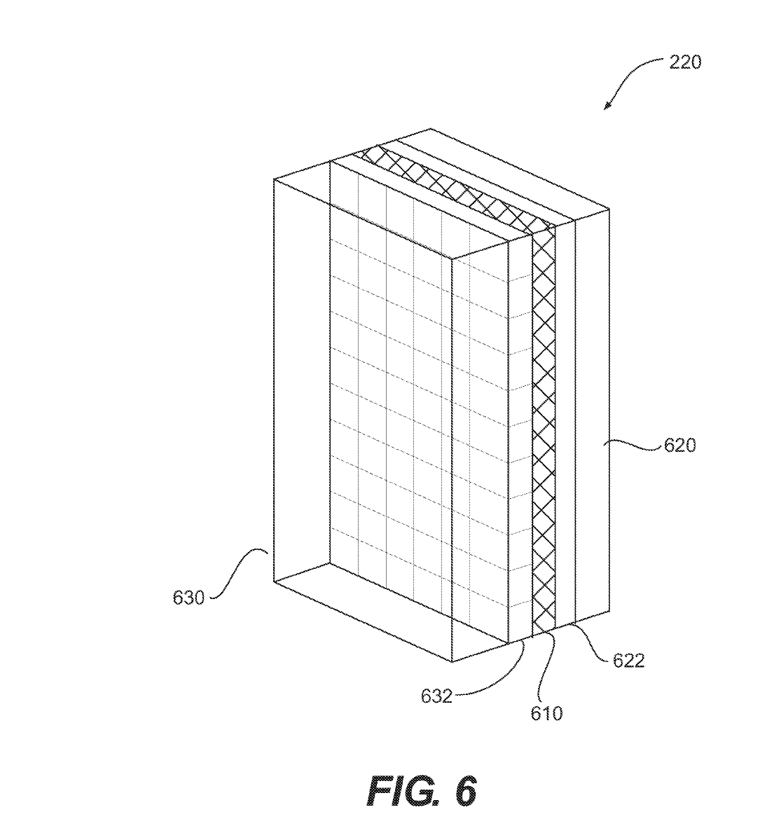

FIG. 6 shows another view of the electro-active element 220 (FIG. 2), which includes an electro-active material 610, such as liquid crystal material, sandwiched between two optical substrates 620 and 630. The thickness of the electro-active material 610 may be between 1 μm and 10 μm, and is preferably less than 5 μm. The substrates 620 and 630 may be substantially flat and parallel, curved and parallel, or one substrate may have a surface relief diffractive pattern and the other substrate may be substantially smooth. The substrates 620 and 630 may provide an optical power or the substrates may have no optical power. Each substrate may have a thickness of 200 μm or less and may be rigid or flexible. Exemplary rigid substrate materials include glass and silicon. Exemplary flexible substrates include flexible plastic films. In general, thinner substrates allows for a higher degree of flexibility for the electro-active element, which may be important for devices that are inserted or implanted into the eye.

A continuous optically transparent electrode 622 that provides for an electrical ground may be disposed on one of the substrates and one or more individually addressable optically transparent electrodes 632 may be disposed on the second substrate. Each electrode 632 defines the size, shape, and/or diameter of a corresponding pixel 642 in the electro-active device. Exemplary pixels may have an area of about 0.25 μm² each with a pixel pitch of about 0.5 μm. Alternatively, pixels may be arranged as concentric rings, arcs, rectangles, or any combination of suitable shapes. One or more of the electrodes 622 and 632 may also form structures that diffract incident light in a fixed pattern or manner. Electrodes 622 and 632 may, for example, comprise a transparent conductive oxide, such as indium tin oxide (ITO), or a conductive organic material, such as poly(3,4-ethylenedioxythiophene) poly(styrenesulfonate) (PEDOT:PSS) or carbon nano-tubes. The thickness of the optically transparent electrodes may be, for example, less than 1 μm, and is preferably less than 0.1 μm. One or more of the electrodes 622 and 632 may be coated with an alignment layer (not shown), with the electro-active material 610 disposed between the alignment layers.

Activating an electrode 632 of combination of electrodes 632 causes respective subsections, or pixels, in the electro-active element 220 to change state. For instance, one or more pixels in the electro-active device may have a transmissivity that varies from about 30% to about 99% in response to an applied voltage. Alternatively, or in addition, one or more pixels in the electro-active device may have a refractive index that varies by up to about 0.1 in response to an applied voltage. The pixel states may be continuous (analog), binary (e.g., transmissive/opaque or high index/low index), or include several discrete values (e.g., 30% transmissive, 50% transmissive, 80% transmissive, etc.). Some electro-active materials, including some liquid crystal materials, remain in active states for only as long as they experience an applied voltage. Other electro-active materials are bi-stable: applying a voltage causes them to switch from one state to another, but no voltage is required to keep them in their current state. Bi-stable electro-active materials are especially attractive for use in implantable ophthalmic device because they consume power only when being switched.

### Using the Electro-Active Element as an Adjustable Aperture

The electro-active element 220 may also be used as an aperture, which is a first region, typically at or near the entrance pupil, that is encompassed by a second region, which may be annular, where the second region has at least one optical characteristic different than the first region. For example, the second region may have a different optical transmission, refractive index, color, or optical path length than the first region. The second region may be referred to as a peripheral region. The optical properties of each region may remain constant within each region, or may vary based on the radius of the region or another function.

One or more of the edges of an aperture provided by the electro-active element 220 may be apodized to alter light entering a wearer's eye. The electro-active element 220 provides the apodized aperture, also known as a mask, by modulating the amplitude, phase, or both of light that is transmitted into the eye through the aperture. Although masks formed with the electro-active element 220 are typically dynamic, the electro-active element 220 can be used to form a static mask that always provide the same modulation of light such as where a static gradient of refractive index or optical transmittance is incorporated into a layer of the device. It can also be used in conjunction with a separate static mask.

For example, the electro-active element 220 can operate as a dynamic aperture that, when closed, increases the depth of field and changes the aggregate optical power of the ophthalmic device 200 by blocking stray light. For example, one or more rings 222 of pixels in the electro-active element 220 may act as or provide an aperture that is about 1.2 mm to about 1.6 mm in diameter when completely closed and about 5.8 mm in diameter when completely open. The aperture can be partially opened such that the diameter varies continuously over the range of about 1.2 mm to about 3.0 mm, e.g., from about 1.2 mm to about 2.5 mm.

Varying the aperture diameter changes the depth of field of the implantable ophthalmic device 200 and increases the effective optical power of the implantable ophthalmic device 200. For an aspheric optical element 210 with a fixed power of 2.0 diopters (D), the average aggregate optical power of the implantable optical device 200 is about 3.5 D when the aperture is 1.2 mm in diameter, about 2.5 D when the aperture is 1.6 mm in diameter, and about 1.3 D when the aperture is 2.0 mm in diameter.

Unfortunately, using the electro-active element 220 alone (i.e., without the aspheric optical element) to increase the depth of field and change the effective optical power also reduces the amount of light transmitted through the implantable ophthalmic device 200. As a result, closing the aperture makes an image projected by the implantable ophthalmic device 200 appear dimmer. If the aperture is too small and/or the ambient light levels are too low, then the image may be too dim to see. Further, the electro-active element 220 by itself cannot provide a broad enough range of optical powers to image objects at intermediate distances (e.g., about 45-100 cm) if it is designed to provide full accommodation for object distances of 50 cm or less. Similarly, static optical elements, including lenses such as the aspheric optical element 210, cannot provide full accommodation at object distances of 50 cm or less without compromising visual acuity at far object distances (e.g., four meters or more).

### Interrelationship Between the Aspheric Optical Element and the Electro-Active Element

Using the aspheric optical element 210 together with the electro-active element 220 makes it possible to provide full accommodation for object distances of 50 cm or less without compromising the patient's ability to image objects at intermediate or far distances. In addition, the shape of the aspheric optical element 210 and/or the states (configurations) of the electro-active element 220 can be chosen such that the implantable ophthalmic device 200 transmits more light than an spherical lens/aperture combination for the same identical optical power and depth of field. In other cases, the electro-active element 220 acts as a diffractive or refractive lens, such as a Fresnel lens, or other element (centered in the aperture) with variable optical power. Changing the size of the aperture and/or the optical power of the electro-active element 210 in response to changes in object distance (estimated from sensor measurements) causes the implantable ophthalmic device's net optical power and depth of field to change so as to provide the best focus for images of near and/or intermediate objects.

For example, an exemplary implantable ophthalmic device can include an aspheric optical element and a spherical optical lens with positive (static) optical power to compensate for a patient's inability to focus on objects at far distances, e.g., four meters or more. The electro-active element may be actuated at object distances from about 30 cm to about 2 m to provide additional optical power for focusing at intermediate object planes (e.g., planes at a distance of about 100 cm) and near object planes (e.g., planes at a distance of about 33-50 cm). In some cases, actuating the electro-active element adds optical power anywhere in a range of about +0.5 D to about +2.5 D, e.g., about +2.0 D. The electro-active element may have discrete settings within the range of optical powers or may be continuously tunable within the range of optical powers.

The performance of exemplary implantable ophthalmic devices with aspheric optical elements and variable-diameter electro-active apertures can be described quantitatively by an optical transfer function (OTF), which is the complex contrast sensitivity function as a function of the spatial frequency of the target object. A complex contrast sensitivity function can be used to characterize the image quality because the optics of the eye may change the spatial frequency of the image relative to that of the target, dependant on the target spatial frequency, in addition to reducing the contrast of the image. In principle, an OTF can be constructed for every object distance and illumination level. The OTF of the eye varies with object distance and illumination level, because both of these variables change the optics of the eye. The OTF of the eye may be reduced due to refractive errors of the eye, ocular aberrations or loss of accommodative ability due to onset of presbyopia.

The image of a point object is the Fourier transform of the aperture convolved with the modulation transfer function (MTF) of the imaging optics, where the MTF is the real component of the OTF discussed above. The resulting point image is known as the point spread function (PSF), and may serve as an index of measurement of the quality of the ocular optic (i.e., a bare eye or eye corrected with a vision care means). The PSF of the retinal image is found to correlate with the quality of visual experience, especially when it is compromised by halos or glint or other image artifacts. Thus, a systematic approach may be applied to selecting an appropriate configuration of the electro-active element.

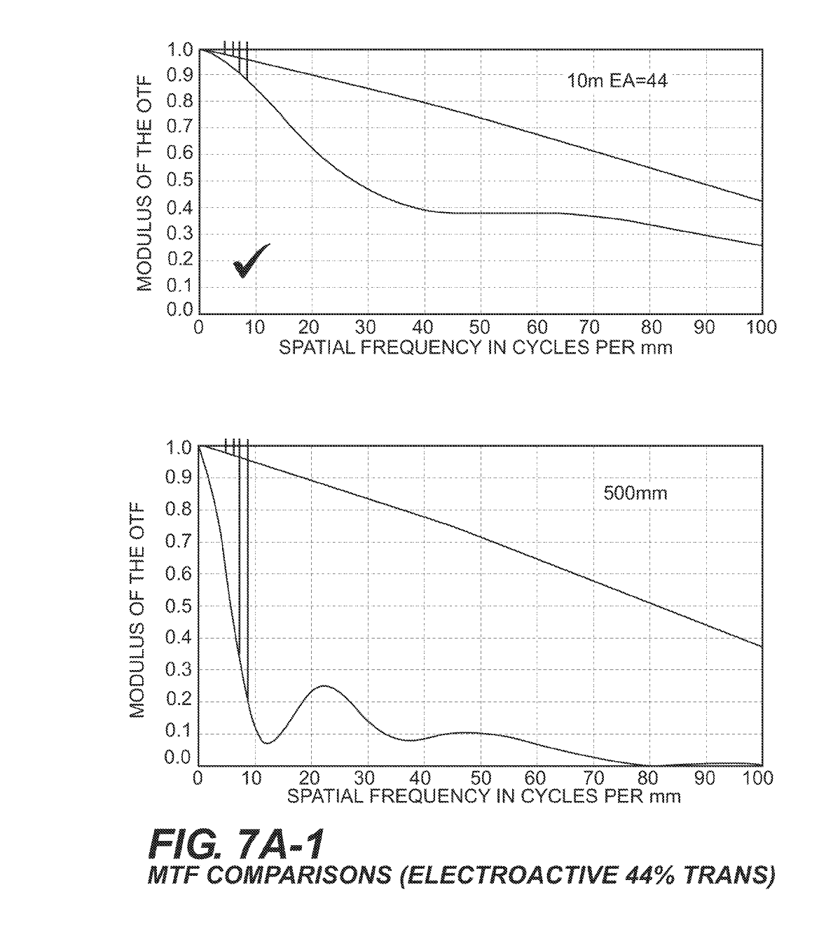

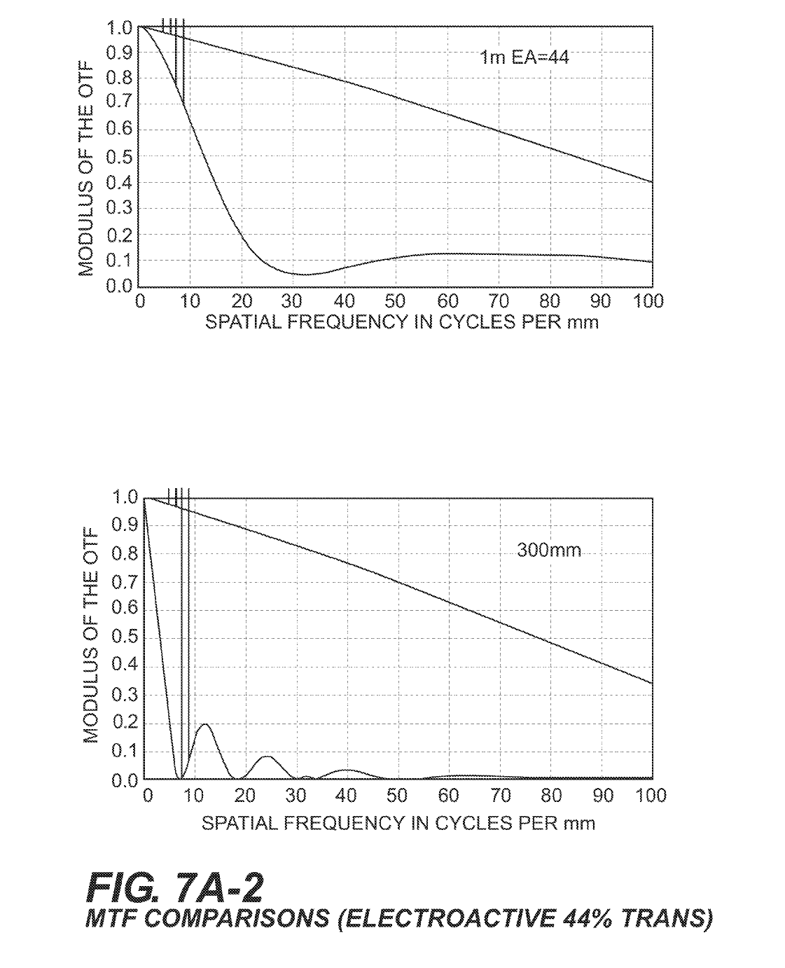

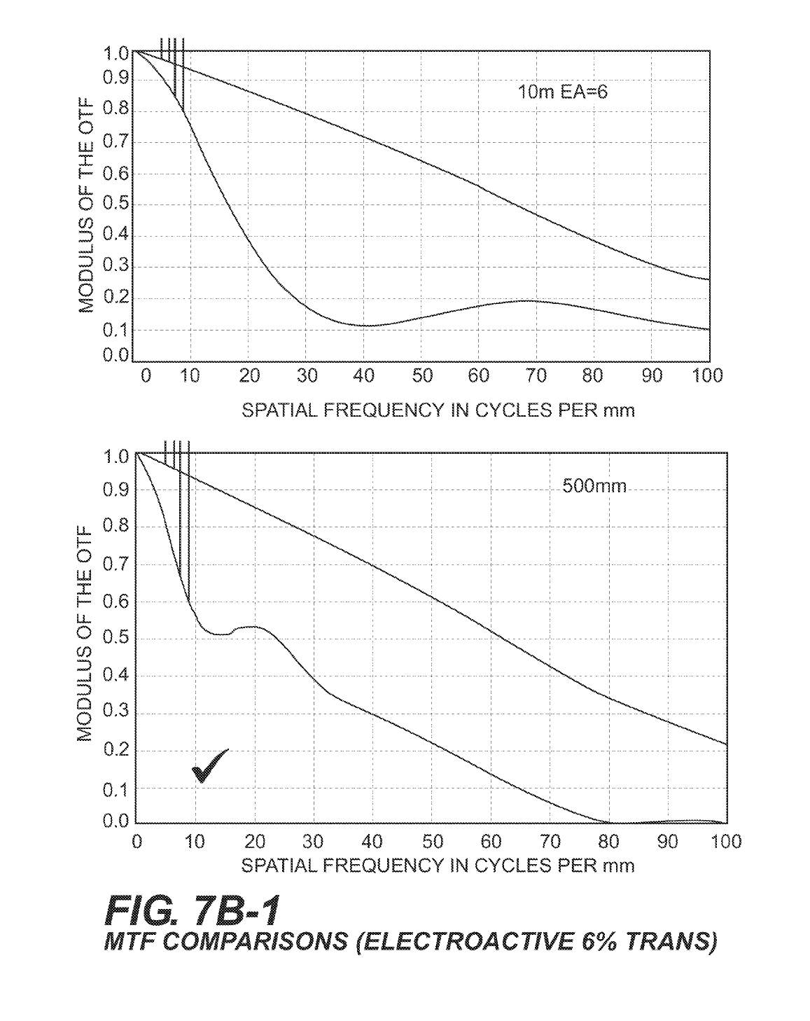

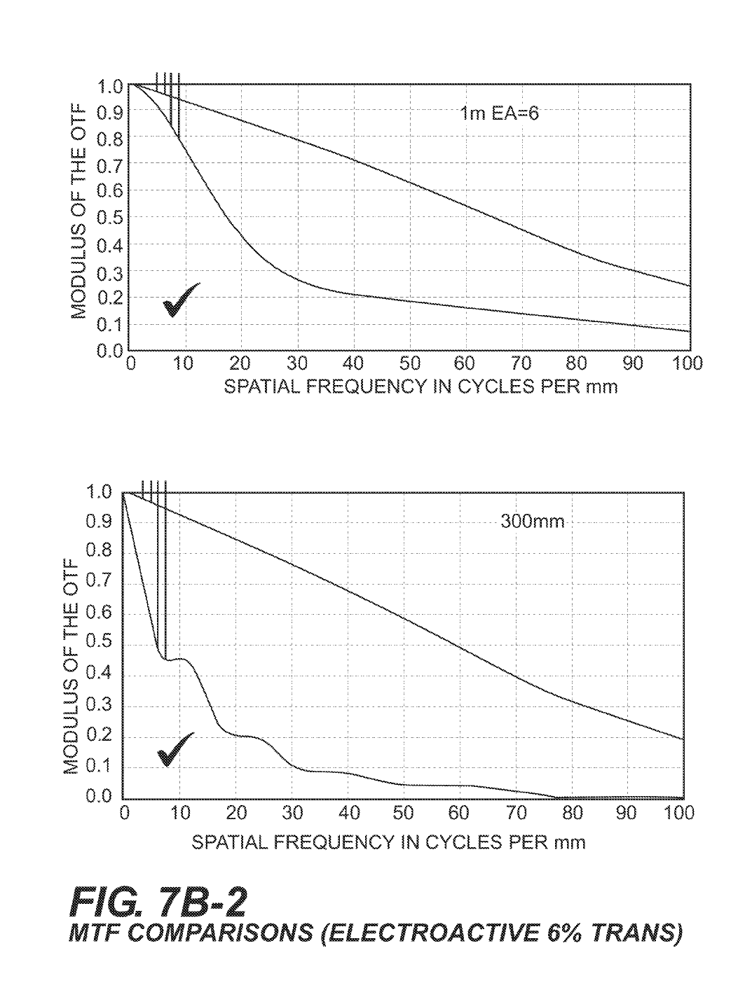

FIGS. 7A and 7B show plots of the MTFs for different object distances of aspheric and standard lenses and used with electro-active apertures with wide diameters (44% transmissive; FIG. 7A) and narrow diameters (6% transmissive; FIG. 7B). As expected, the MTFs and the image quality for the aspheric lens/aperture combinations is significantly better than those for the standard lens/aperture combinations. The MTF for each aspheric lens/aperture combination trails off with approximately linear slope, whereas the MTF for each standard lens/aperture combination exhibits underdamped oscillation before approaching zero. The variation among the MTFs for the aspheric lens/aperture combinations is also much smaller than the variation among the MTFs for the standard lens/aperture combinations.

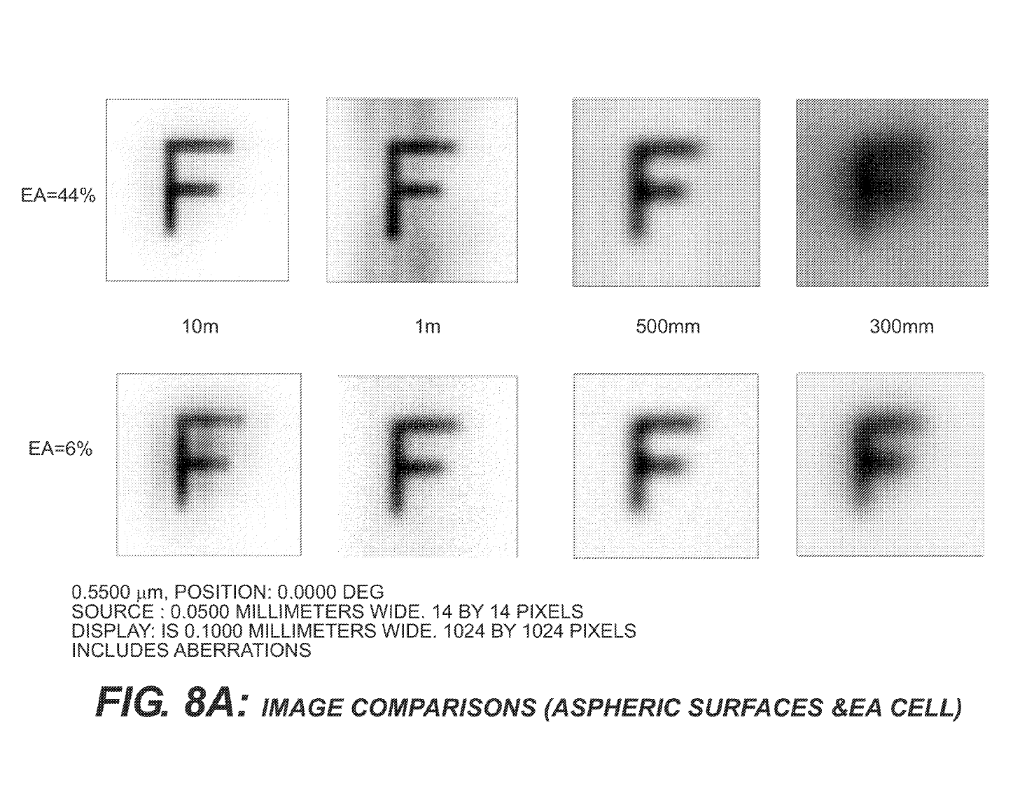

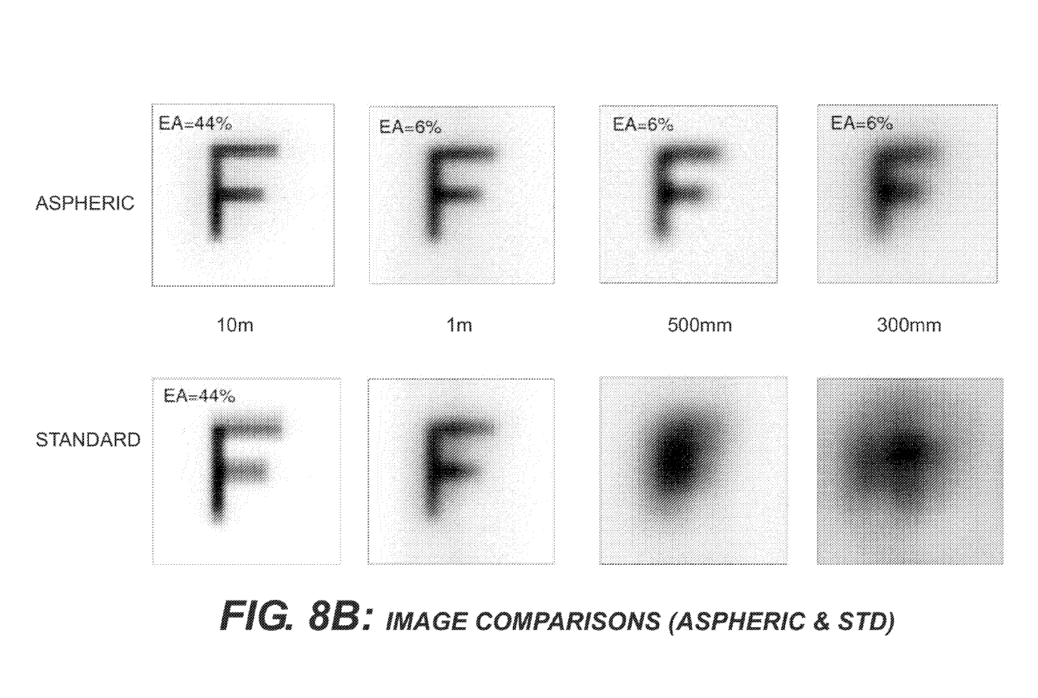

FIG. 8A shows images captured with the aspheric lens of FIGS. 7A and 7B in combination with an electro-active aperture that is open to a 44% transmissive state (top row) and closed to a 6% transmissive state (bottom row). As expected, the image quality for far objects (3 m) is better for wider apertures, and the image quality for near objects is better for narrower apertures. FIG. 8B show images captured with the lens/aperture combinations of FIGS. 7A and 7B. Again, the aspheric lens/aperture combination produces sharper images than the standard lens/aperture combinations for every object distance.

There is a trade-off between the plus power added by the dynamic element and the static element, the rate of change of power contributed by the static element as a function of pupil size, and the object distance (which is inferred from the pupil size) at which the electro-active element is activated. TABLE 2 illustrates this trade-off by showing the blur of an image seen through an implanted ophthalmic device for different object distances (and pupil diameters) when the electro-active element is switched on at object distances of 100 cm, 50 cm, and 40 cm. In this example, the aspheric optical element has a peak optical power of 0.75 D at is center, and the electro-active element contributes an optical power of 2.50 D when switched on. An image blur of 0.50 D for objects at a distance of 200 cm is equal to the natural depth of field. TABLE 2 shows that when the electro-active element is designed to deliver 2.50 D and the static element has a maximum power of 0.75 D, the implanted ophthalmic device exhibits optimum performance when the electro-active element is switched on at an object distance of about 50 cm.

TABLE 2
Image Blur for Different Electro-
Active Element Switching Distances

| | Assumed | Image Blur (D) for an Electro-Active | | |
|---|---|---|---|---|
| Object | Pupil | Element switched on at: | | |
| Distance | Diameter | 100 cm Object | 50 cm Object | 40 cm Object |
| (cm) | (mm) | Distance | Distance | Distance |
| 200 | 4.5 | 0.50 | 0.50 | 0.50 |
| 100 | 4.0 | 1.65 | 0.75 | 0.75 |
| 75 | 3.8 | 1.45 | 1.05 | 1.05 |
| 50 | 3.5 | 0.85 | 0.85 | 1.15 |
| 33 | 3.0 | 0.0 | 0.0 | 0.0 |
| 30 | 2.9 | 0.15 | 0.15 | 0.15 |
| 25 | 2.5 | 0.75 | 0.75 | 0.75 |

The optimum switching configuration depends on the plus power delivered by the electro-active element and the design of the aspheric optical element. In general, the magnitude of defocus at intermediate object distances increases as the plus power delivered by the electro-active element increases.

### Examples of Processors for Use in Implantable Ophthalmic Device

As described above, exemplary implantable ophthalmic devices may include a processor electrically coupled to the electro-active element and configured to actuate electro-active element in response to indications of pupil size and/or user input. In some examples, the processor is a integrated circuit, such as ASIC, that includes memory for storing one or more settings for driving the electro-active element. When the processor receives an indication that the user is trying to focus on objects at a different distance, e.g., as shown by a change in pupil size, it switches one or more pixels in the electro-active element by applying voltages to the appropriate electrodes. The processor (and associated software or firmware) may be capable of arbitrarily addressing multiple segments in a preprogrammed or adaptable manner. Associated software and/or firmware may be permanently embodied in a computer-readable medium, such as a special-purpose chip or a general purpose chip that has been configured for a specific use, or it may be provided by an analog or digital signal.

The processor can be programmed to actuate the electro-active element based on a particular (or representative) patient's pupil size, which varies as a function of depth of field, patient age, patient race, patient weight, etc. A specialist, such as an ophthalmologist, chooses the optimum settings of the electro-active element for achieving the desired vision performance based on the merit function, then uploads the settings to the ophthalmic device via an antenna, such as a light-sensitive cell or an rf antenna. The device may be programmed before implantation and/or after implantation. In some cases, the device may be reprogrammed on a regular basis, e.g., annually, based on changes in the patient's vision and/or natural degradation of the device itself. The ophthalmologist may also use the patient's feedback to adjust the settings of electro-active element as appropriate.

### Examples of Sensors for Use in Implantable Ophthalmic Device

A sensor may be used to measure or infer the distance to the object(s) that the user is trying to focus on. The sensor may be operably (e.g., wirelessly or electrically) coupled to processor and may provide an indication of the object distance and/or pupil size to the processor. The sensor may include one or more sensing elements, such as a range finder for detecting a distance to which a user is trying to focus and/or a light-sensitive cell for detecting light that is ambient and/or incident to the implantable ophthalmic device. Suitable light-sensitive cells include, but are not limited to photodetectors, photovoltaic cells, and ultraviolet- or infrared-sensitive photo cells. Other suitable sensing elements include, but are not limited to a tilt switch, a passive range-finding device, a time-of-flight range finding device, an eye tracker, a view detector, an accelerometer, a proximity switch, a physical switch, a manual override control, a capacitive switch that switches when a user touches the nose bridge of a pair of spectacles, a pupil diameter detector, a bio-feed back device connected to an ocular muscle or nerve, or the like. The sensor may also include one or more micro electro mechanical system (MEMS) gyroscopes adapted for detecting a tilt of the user's head or encyclorotation of the user's eye.

An illustrative sensor may include two or more photo-detector arrays with a focusing lens placed over each array. Each focusing lens may have a focal length appropriate for a specific distance from the user's eye. For example, three photo-detector arrays may be used, the first one having a focusing lens that properly focuses for near distance, the second one having a focusing lens that properly focuses for intermediate distance, and the third one having a focusing lens that properly focuses for far distance. A sum of differences algorithm may be used to determine which array has the highest contrast ratio (and thus provides the best focus). The array with the highest contrast ratio may thus be used to determine the distance from a user to an object the user is focusing on.

When the sensor detects changes in object distance, pupil size, and/or intensity, it sends a signal to the processor which triggers the activation and/or deactivation of the electro-active element in the implantable ophthalmic device. For example, the sensor may detect the intensity of light and communicate this information to the processor. If the sensor detects that a user is focusing within a near distance range, the processor may cause the electro-active element to increase its optical power. If the sensor detects that the user is focusing beyond the near distance range, the processor may cause the electro-active element to decrease its optical power. The processor may have a delay feature which ensure that a change in intensity of light is not temporary (i.e., lasts for more than the delay of the delay feature). Thus, when a user blinks his or her eyes, the size of the aperture will not be changed since the delay of the delay circuit is longer than the time it takes to blink. The delay may be longer than approximately 0.0 seconds, and is preferably 1.0 seconds or longer.

Some configurations may allow for the sensor and/or processor to be overridden by a manually operated remote switch. The remote switch may send a signal by means of wireless communication, acoustic communication, vibration communication, or light communication such as, by way of example only, infrared. By way of example only, should the sensor sense a dark room, such as a restaurant having dim lighting, the controller may cause the dynamic aperture to dilate to allow more light to reach the retina. However, this may impact the user's ability to perform near distance tasks, such as reading a menu with small print. For instance, the user could remotely control the electro-active element of the implantable ophthalmic device to change the optical power and/or to increase the depth of field and enhance the user's ability to read the menu. When the near distance task has completed, the user may remotely allow the sensor and controller to cause the electro-active element to revert back to its previous optical power and/or depth of field settings. For more on electrical, optical, and mechanical sensors, see U.S. Pat. No. 7,926,940 to Blum et al., which is incorporated herein by reference in its entirety.

Alternatively, the sensor can include an electrochemical detector that monitors the changes in ion concentration in the eye, e.g., in the ocular cytosolic fluid. As understood by those skilled in the art, the accommodative response (also known as the accommodative loop) includes at least three involuntary ocular responses: (1) ciliary muscle contraction, (2) iris sphincter contraction (pupil constriction increases depth of focus), and (3) convergence (looking inward enables binocular fusion at the object plane for maximum binocular summation and best stereoscopic vision). Both the ciliary muscle and the iris sphincter are smooth muscles whose relaxation and contraction is regulated by an ion channel that carries calcium, sodium, potassium, phosphate, magnesium, zinc, or any other suitable ion. When an accommodative impulse causes the ciliary muscle and/or the iris sphincter relax and/or contract, the ion concentration in the ion channel changes by amount or differential that can be measured by the electrochemical detector, which emits an electrical signal in response to the change in ion concentration. For more on accommodative triggers and sensors, see U.S. application Ser. No. 12/496,838 to Gupta et al., entitled “Sensor for Detecting Accommodative Trigger” and filed on Jul. 2, 2009, which is incorporated herein by reference in its entirety.

### Examples of Antennas for Use in Implantable Ophthalmic Device

The device 200 also may include an antenna 260, which may be configured to receive either radio-frequency (rf) or optical signals for controlling or updating the processor 220, and may also be used to charge the batteries 250 as described below. The antenna may also receive manual override signals.

### Examples of Power Supply for Use in Implantable Ophthalmic Device

The processor may draw at least some of its electrical power from a power supply, such as a capacitor or thin-film rechargeable battery like those manufactured by Excellatron. For example, one or more rechargeable batteries 250 coupled to the processor 220 provide power for the processor 220 and other electronic components in the implantable ophthalmic device 200 as shown in FIG. 2. Thin-film rechargeable batteries may be capable of being cycled in excess of 45,000 cycles, which could translate to a usable lifetime of 20-25 years in the lens or optic. Two thin film rechargeable batteries may be used and may stacked one atop the other. In this configuration, one of the batteries may be used for 20-25 years and the other battery may be switched to when the first battery is no longer operable. Alternatively, the other battery may be switched to by a signal sent remotely to the controller. This may extend the lifetime of the optic or lens to 40-50 years. The power supply may be remotely charged, by way of example only, by induction, as explained in U.S. application Ser. No. 12/465,970 entitled “Device for Inductive Charging of Implanted Electronic Devices,” which is incorporated herein by reference in its entirety.

One or more light-sensitive cells, such as solar cells or photovoltaic cells, may also be used to supplement, augment, and/or obviate the need for a battery. The light-sensitive cell is located out of the user's line of sight of the user, e.g., peripheral to the margin of the pupil when partially dilated by darkness, but not fully dilated. The device may thus be charged by using an eye-safe laser capable of energizing the light-sensitive cell or cells.

Alternatively, the light-sensitive cell may be located in front of (closer to the cornea of the eye) and separately disposed from a portion of the iris of a user's eye. Thin electrical wiring may operably connect the solar cell to the processor. The electrical wiring may pass through the pupil without touching the iris and operably connect to the implantable ophthalmic device. The solar cell may be large enough such that it supplies enough electrical power to obviate the need for a separate power supply. The thin electrical wiring may not conduct electricity and may have a form factor which has the appropriate tensile strength to hold the solar cell in place. In some configurations, one or more small holes may be made in the iris by an ophthalmic laser such that the thin electrical wiring connects the solar cell to the implantable ophthalmic device.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. An implantable ophthalmic device comprising:
an aspheric optical element having a negative spherical aberration that varies as a function of radius.

2. The implantable ophthalmic device of claim 1 wherein the negative spherical aberration is at a maximum at or near the optical center of the aspheric optical element.

3. The implantable ophthalmic device of claim 1 wherein the negative spherical aberration varies within a range of about 0.10 μm to about 5.0 μm of root-mean-square wavefront error across an exit pupil of about 5.0 mm or less in diameter.

4. The implantable ophthalmic device of claim 1 wherein the negative spherical aberration is substantially non zero over a radius about 0.50 mm to about 2.5 mm centered about the geometric center of the aspheric optical element.

5. The implantable ophthalmic device of claim 1 wherein the aspheric optical element has a sag that is continuous.

6. The implantable ophthalmic device of claim 4 wherein a first derivative of the sag with respect to the transverse dimensions is continuous.

7. The implantable ophthalmic device of claim 5 wherein a second derivative of the sag with respect to the transverse dimensions is continuous.

8. The implantable ophthalmic device of claim 1 wherein the aspheric optical element, when implanted in a patient's eye, provides an average incremental optical power of about +0.25 D or less.

9. The implantable ophthalmic device of claim 7 wherein the aspheric optical element, when implanted in a patient's eye, provides an average incremental optical power of about +0.10 D or less.

10. The implantable ophthalmic device of claim 1 wherein the aspheric optical element, when implanted in a patient's eye, provides a maximum incremental optical power of about +0.5 D to about +0.8 D.

11. The implantable ophthalmic device of claim 1 further comprising:
a spherical optical element in optical communication with the aspheric optical element.

12. The implantable ophthalmic device of claim 16 wherein the spherical optical element has an optical power of about +10 D to about +36 D.

13. The implantable ophthalmic device of claim 1 further including an electro-active element in optical communication with the aspheric optical element and having a first state with a first effective optical power and a second state with a second effective optical power.

14. The implantable ophthalmic device of claim 13 wherein the electro-active element has a first refractive index in the first state and a second refractive index in the second state.

15. The implantable ophthalmic device of claim 13 wherein the electro-active element has a first transmissivity in the first state and a second transmissivity in the second state.

16. The implantable ophthalmic device of claim 13 wherein the electro-active element provides an aperture with a first diameter in the first state and a second diameter in the second state.

17. The implantable ophthalmic device of claim 13 wherein the electro-active element provides at least one of the first and second effective optical powers is about +0.5 D to about +2.5 D.

18. The implantable ophthalmic device of claim 13 further comprising:
a processor operably coupled to the electro-active optical element and configured to actuate the electro-active optical element.

19. The implantable ophthalmic device of claim 18 further comprising:
a sensor operably coupled to the processor and configured to provide an indication of pupil size to the processor, and
wherein the processor is configured to actuate the electro-active optical element in response to the indication of the pupil size.

20. The implantable ophthalmic device of claim 18 further comprising:
an antenna operably coupled to the processor and configured to transmit and receive data

21. The implantable ophthalmic device of claim 20 further comprising:
at least one battery operably coupled to the processor and configured to provide power to the processor, and
wherein the battery is configured to be recharged via the antenna.

22. An implantable ophthalmic device comprising:
a spherical optical element having a fixed optical power;
an aspheric optical element in optical communication with the spherical optical element, the aspheric optical element having an optical power that varies as a function of radius; and
an electro-active element in optical communication with the spherical and aspheric optical elements, the electro-active element having a first state with a first effective optical power and a second state with a second effective optical power.

23. The implantable ophthalmic device of claim 21 wherein the fixed optical power is from about +10 D to about +36 D.

24. The implantable ophthalmic device of claim 21 wherein the aspheric optical element, when implanted in a patient's eye, provides an average incremental optical power of about +0.25 D or less.

25. The implantable ophthalmic device of claim 23 wherein the aspheric optical element, when implanted in a patient's eye, provides an average incremental optical power of about +0.10 D or less.

26. The implantable ophthalmic device of claim 21 wherein the aspheric optical element, when implanted in a patient's eye, provides a maximum incremental optical power of about +0.5 D to about +0.8 D.

27. The implantable ophthalmic device of claim 21 wherein at least one of the first and second effective optical powers is about +0.5 D to about +2.5 D.

28. An implantable ophthalmic device comprising:
a spherical optical element;
an aspheric optical element in optical communication with the spherical optical element, the aspheric optical element having a sag whose first and second derivatives with respect to radius are continuous; and
an electro-active element in optical communication with the spherical and aspheric optical elements, the electro-active element having a first state with a first effective optical power and a second state with a second effective optical power.

29. The implantable ophthalmic device of claim 28 wherein the aspheric optical element has a negative spherical aberration that varies as a function of radius.

30. The implantable ophthalmic device of claim 29 wherein the negative spherical aberration is at a maximum at or near the optical center of the aspheric optical element.

31. The implantable ophthalmic device of claim 29 wherein the negative spherical aberration varies within a range of about 0.10 μm to about 5.0 μm of root-mean-square wavefront error across an exit pupil of about 5.0 mm or less in diameter.

32. The implantable ophthalmic device of claim 29 wherein the negative spherical aberration is substantially non zero over a radius about 0.50 mm to about 2.5 mm centered about the geometric center of the aspheric optical element.
